(19) United States
(12) Patent Application Publication
(10) Pub. No.: US 2026/0331296 A1
(43) Pub. Date: Oct. 22, 2026

(54) SYSTEM AND METHOD FOR ANALYZING RNA-SEQUENCING (RNA-SEQ) DATA TO DETECT EXPRESSION ABERATIONS AND SYNTHETIC RNA INTERFERENCE

(71) Applicant: Neo7Bioscience, Inc., Dallas, TX (US)

(72) Inventor: John A. Catanzaro, Rockwall, TX (US)

(21) Appl. No.: 19/649,670

(22) Filed: Apr. 16, 2026

Related U.S. Application Data
(60) Provisional application No. 63/789,629, filed on Apr. 16, 2025.

(51) Int. Cl. G16H 50/30; G16B 20/20; G16B 30/00; G16B 40/10; G16B 40/20; G16H 15/00

(52) U.S. Cl. CPC G16H 50/30; G16B 20/20; G16B 30/00; G16B 40/10; G16B 40/20; G16H 15/00

(57) ABSTRACT

Provided for herein are systems and methods for analyzing RNA-sequencing (RNA-seq) data to detect expression aberrations and synthetic RNA interference. The processor is configured to receive RNA-sequencing data related to a human subject; identify a signal detection in the RNA-sequencing data; screen the signal detection for an expression index; calculate a risk score using the RNA-sequencing data, the signal detection, and the expression index; and display the risk score on a graphical user interface.

### CROSS-REFERENCE TO RELATED APPLICATIONS

This application claims the benefit of U.S. Provisional Application No. 63/789,629 filed on Apr. 16, 2025, and entitled “REVISS: A SCREENING SIGNAL ALERT TOOL AND PROGNOSTIC FRAMEWORK FOR RNA EXPRESSION VARIANT INSTABILITY SURVEILLANCE” the entirety of which is incorporated by reference in its entirety.

### FIELD OF THE INVENTION

The preset invention generally relates to the field of bioinformatics, molecular diagnostics, and computational pathology. In particular, the present invention is directed to a system and method for analyzing RNA-sequencing (RNA-seq) data to detect expression aberrations and synthetic RNA interference in human samples.

### BACKGROUND

RNA-seq technologies are widely used in clinical and research settings for profiling gene expression. However, traditional approaches focus primarily on canonical differential expression without assessing the instability patterns, foreign synthetic elements, or variant-linked oncogenic drivers. As mRNA-based biologics, environmental RNA fragments, and synthetic RNA contaminants become increasingly common, there is a growing need for systems that can surveil and quantify these risks with precision.

### SUMMARY

In an aspect, a system for analyzing RNA-sequencing (RNA-seq) data to detect expression aberrations and synthetic RNA interference, the system comprising at least a processor; and a memory communicatively connected to the at least a processor, the memory containing instructions configuring the at least a processor to receive RNA-sequencing data related to a human subject; identify a signal detection in the RNA-sequencing data; screen the signal detection for an expression index; calculate a risk score using the RNA-sequencing data, the signal detection, and the expression index; and display, the risk score on a graphical user interface.

In yet another non-limiting aspect, a method of analyzing RNA-sequencing (RNA-seq) data to detect expression aberrations and synthetic RNA interference, the method comprising receiving, RNA-sequencing data related to a human subject; identifying, a signal detection in the RNA-sequencing data; screening, the signal detection for an expression index; calculating, a risk score using the RNA-sequencing data, the signal detection, and the expression index; and displaying, the risk score on a graphical user interface.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for analyzing RNA-sequencing (RNA-seq) data to detect expression aberrations and synthetic RNA interference;

FIG. 2 is a block diagram illustrating an exemplary embodiment of a machine-learning process;

FIG. 3 is a block diagram illustrating an exemplary embodiment of a neural network;

FIG. 4 is a block diagram illustrating an exemplary embodiment of a node of a neural network;

FIG. 5 is a block diagram illustrating an exemplary embodiment of a computing system that can be used to implement any one or more of the methodologies disclosed herein any one or more portions thereof;

FIG. 6 is a process flow diagram illustrating an exemplary embodiment of a method of analyzing RNA-sequencing (RNA-seq) data to detect expression aberrations and synthetic RNA interference;

FIG. 7A is a graphical diagram illustrating an exemplary embodiment of a transcriptomic profile;

FIG. 7B is a graphical diagram illustrating an exemplary embodiment of a transcriptomic profile; and

FIG. 8 is a graphical diagram illustrating an exemplary embodiment of a risk score.

### DETAILED DESCRIPTION

At a high level, aspects of the present disclosure are directed to systems and methods for analyzing RNA-sequencing (RNA-seq) data to detect expression aberrations and synthetic RNA interference.

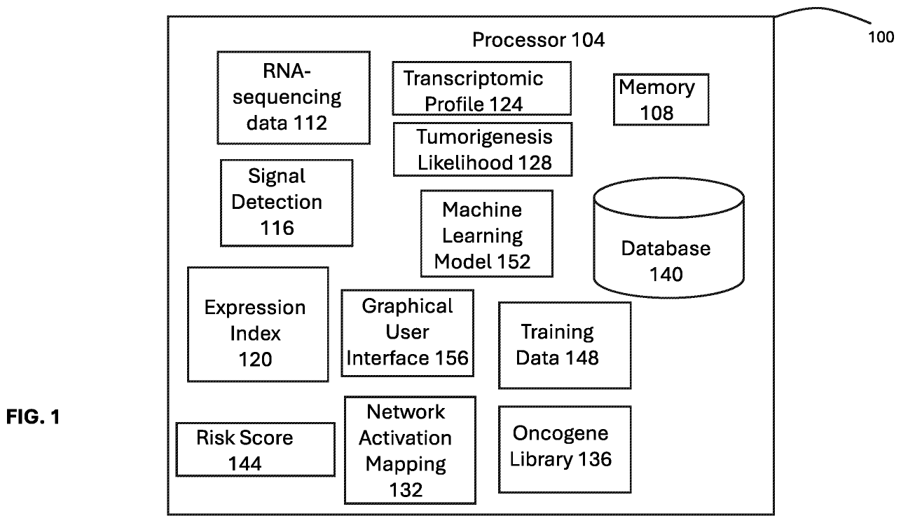

Referring now to FIG. 1, system 100 includes at least a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. processor may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device, processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of system 100 and/or computing device. A network configuration may include multihop, mesh, tree, start ring, fully connected, bus, and/or one or more ad hoc networks. Network may include one or more features including but not limited to electromagnetic, sound, radio, and optical. Network may operate in line of sight or long range.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks, processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory 108. Memory 108 may include various components (e.g., machine-readable media) including, but not limited to, a random-access memory component, a read only component, and any combinations thereof. In one example, a basic input/output system (BIOS), including basic routines that help to transfer information between elements within apparatus 100, such as during start-up, may be stored in memory. Memory 108 may also include (e.g., stored on one or more machine-readable media) instructions (e.g., software) embodying any one or more of the aspects and/or methodologies of the present disclosure. In another example, memory may further include any number of program modules including, but not limited to, an operating system, one or more application programs, other program modules, program data, and any combinations thereof.

With continued reference to FIG. 1, the at least a processor is configured to receive RNA-sequencing data 112 related to a human subject. “RNA-sequencing data” as used in this disclosure is a collection of sequencing reads generated from RNA molecules to quantify and identify RNA molecules in a biological sample. RNA-sequencing data 112 may consist of short DNA sequences that originate from RNA molecules after they've converted into complementary DNA (cDNA) and sequenced using next-generation sequencing technologies. These reads reveal which RNA molecules are present and in what quantities. RNA-sequencing data 112 may reveal gene expression levels indicating how much of each gene is transcribed. RNA-sequencing data 112 may include alternative splicing including different transcript isoforms from the same gene. RNA-sequencing data 112 may include an identification of RNA types present, including but not limited to mRNA, miRNA, tRNA, rRNA and the like. RNA-sequencing data 112 may include mutations, single nucleotide polymorphisms (SNPs), gene fusions, and other transcriptomic variations. RNA-sequencing data 112 may include dynamic cellular states since RNA levels may change rapidly with environment, disease, or cell type.

With continued reference to FIG. 1, RNA-sequencing data 112 may be obtained from a human subject. A “human subject” as used in this disclosure, is any member of the species Homo sapiens.

With continued reference to FIG. 1, RNA-sequencing data 112 may be selected from one or more genes associated with immune regulation, mitochondrial function, cell cycle, inflammation, and DNA repair. For instance and without limitation, genes associated with immune regulation may include cytokine and signaling genes such as IL family genes, TNF/TNFR genes, IFN pathway genes, and JAK/STAT pathway components. Genes associated with immune regulation may include antigen presentation genes including HLA class I and II genes, TAP½, and B2M. Genes associated with immune regulation may include T-cell and B-cell regulatory genes including ROXP3 (T-reg master regulator), CTLA4, PDCD1 (PD-1), CD28, and CD40/CD40L. Genes associated with immune regulation may include innate immune regulators such as TLR family genes, NLRP3 and other inflammasome components, and complement genes. Genes associated with immune regulation may include transcription factors and epigenetic regulators including NF-kB family, IRF family, STAT family, and chromatin modifiers.

With continued reference to FIG. 1, genes associated with mitochondrial function may include complex I including ND1, ND2, ND3, ND4, ND4L, ND5, and ND6. Genes associated with mitochondrial function may include Complex III including CYB. Genes associated with mitochondrial function may include Complex IV including COX1, COX2, and COX3. Genes associated with mitochondrial function may include Complex V including ATP6 and ATP8. Genes associated with mitochondrial function may include genes for respiratory chain assembly factors, mitochondrial ribosomal proteins, protein import machinery, mitochondrial DNA replication and repair, metabolic enzymes, and mitochondrial signaling and stress-response pathways. Genes associated with mitochondrial function may include POLG, POLG2, TFAM, TOMM/TIMM family, NDUFS/NDUFA families, SDHA/B/C/D, UQCRC½, COX4I1, COX5A/B, ATP5F1A/B/C, PGC-1 α (PPARGCIA), OPA1, MFN½, and DRP1. Genes associated with mitochondrial function may include genes associated with mitochondrial signaling and stress responses including for example NFR1, NRF2, SIRT3, SIRT4, SIRT5, HSP60, and HSP10.

With continued reference to FIG. 1, RNA-sequencing data 112 may include genes associated with the cell cycle, including cyclins such as CCNA1, CCNA2, CCNB1, CCNB2, CCNB3, CCND1, CCND2, CCND3, CCNE1, CCNE2. Genes associated with the cell cycle may include cyclin dependent kinases including CDK1, CDK2, CDK4, CDk6, and CDK7. Genes associated with the cell cycle may include CDK inhibitors including CDKNIA (p21), CDKNIB (p27), CDKN2A (p16), CDKN2B, CDKN2C, and CDKN2D. Genes associated with the cell cycle may include checkpoint and DNA damage response genes including ATM, ATR, CHEK1, CHEK2, TP53, and CDC25A/B/C. Genes associated with the cell cycle may include anaphase promoting complex and cyclosomes including ANAPC1, ANAPC2, ANAPC4, ANAPC5, ANAPC7, ANAPC10, ANAPC11, and ANAPC13. Genes associated with the cell cycle may include core cell division genes including CDC6, CDC7, CDC20, CDC23, CDC27, CDC45, BUB1, BUB1B, BUB3, CENPF, KIF23, PLK1, TOP2A. Genes associated with the cell cycle may include transcription factors controlling cell cycle gene expression including E2F1, E2F2, E2F3, E2F4, E2F5, ZNF519, ZNF274, and RB1.

With continued reference to FIG. 1, RNA-sequencing data 112 may include genes associated with inflammation including pro-inflammatory cytokine genes such as IL1B, IL1A, IL6, TNF α, IL18, IL12A, IL12B, IL23A, IFNB1, and IFNA. Genes associated with inflammation may include chemokind genes including CXCL8, CCL2, CCL5, and CXCL10. Genes associated with inflammation may include pattern-recognition receptors including TLR family (TLR2, TLR4, TLR7, and TLR9), NOD1, NOD2, RIG-I (DDX58), MDA5 (IFIH1), cGAS (MB21D1), and STING (TMEM173). Genes associated with inflammation may include inflammasome sensor genes including NLRP3, NLRP1, NLRC4, MEFV (pyrin), and AIM2. Genes associated with inflammation may include inflammasome adaptor and effector genes including PYCARD (ASC), CASP1, CASP4, CASP5, and NEK7. Genes associated with inflammation may include NF-kB pathway genes including NF-kB1, NF-kB2, RELA, RELB, IKBKB, and IKBKG (NEMO). Genes associated with inflammation may include JAK-STAT pathway genes including JAK1, JAK2, TYK2, STAT1, STAT2, and STAT3. Genes associated with inflammation may include anti-inflammatory and regulatory genes IL10, IL1RN, SOCS1, SOCS3, TNFAIP3 (A20). Genes associated with inflammation may include genes implicated in human inflammatory diseases including HLA genes, IFIH1, ERAP1, SIRPB1, BLK, and FAM167A.

With continued reference to FIG. 1, RNA-sequencing data 112 may include genes associated with DNA repair including mismatch repair genes such as MLH1, MSH2, MSH6, PMS2, and EPCAM. Genes associated with DNA repair may include nucleotide excision repair including XPA, XPB (ERCC3), XPC, XPD (ERCC2), XPE (DDB2), XPF (ERCC4), XPG (ERCC5), CSA (ERCC8), CSB (ERCC6), ELOF1, and STK19. Genes associated with DNA repair may include base excision repair (BER) including OGGI, MUTYH, APE1 (APEX1), POLB, XRCC1, PARP1, and PARP2. Genes associated with DNA repair may include homologous recombination (HR) including BRCA1, BRCA2, RAD51, RAD51C, RAD51D, XRCC2, XRCC3, PALB2, ATM, HROB, MCM8, and MCM9. Genes associated with DNA repair may include non-homologous end joining (NHEJ) including KU70 (XRCC6), KU80 (XRCC5), DNA-PKcs (PRKDC), XRCC4, LIG4, NHEJ1 (XLF), 53BP1, FIF1, SHLD1-3, and ERCC6L2. Genes associated with DNA repair may include Fanconi anemia pathway genes including FANCA, FANCB, FANCC, FANCD1 (BRCA2), FANCD2, FANCE, FANCF, FANCG, FANCI, FANCJ (BRIP1), FANCL, FANCM, FANCN (PALB2), FANCO (RAD51C), FANCP (SLX4), FANCQ (ERCC4), FANCR (RAD51), FANCS (BRCA1), FANCT (UBE2T), FANCU (XRCC2), FANCV (REV7). Genes associated with DNA repair may include direct reversal repair including MGMT, ALKBH2 and ALKBH3. Genes associated with DNA repair may include DNA damage signaling and checkpoint genes including ATM, ATR, CHEK1, CHEK2, TP53, H2AX (H2AFX), and PARP1. Genes associated with DNA repair may include PRIMOL, ATAD5 (ELG1), DSCC1, CHTF8, TXNDC17, CYB5R4, ZATT, and TDP2.

With continued reference to FIG. 1, the at least a processor 104 is configured to identify a signal detection 116 in the RNA-sequencing data 112. A “signal detection” as used in this disclosure, is the identification of any synthetic or exogenous RNA motifs, including but not limited to spike protein derived sequences, poly-A tail irregularities, and synthetic 5′ cap analogs. An RNA motif may include a short, structured pattern within an RNA molecule that carries a specific biological meaning. An RNA motif may act as an instructional symbol that informs RNA how to behave, including how long it should last, where it should go, how it should fold, and what proteins it should interact with. An RNA motif may include a sequence motif which contains a specific nucleotide pattern. An RNA motif may include a structural motif that contains shapes formed by folding. An RNA motif may include a functional motif which contains regions that recruit proteins or regulate translation. An RNA motif may influence stability such as how quickly RNA is degraded. An RNA motif may influence translation efficiency indicating how much protein is made. An RNA motif may influence localization where in the cell the RNA travels. An RNA motif may influence immune recognition to determine whether the immune system sees the RNA as “self” or “foreign.” An RNA motif may influence splicing such as how exons may be included or excluded. An RNA motif may include but is not limited to a Poly(A) tail signal, a 5′ cap structure, AU rich elements (AREs), internal ribosome entry sites (IRES), miRNA binding sites, and stem-loops and hairpin structural motifs used in regulation and viral replication.

With continued reference to FIG. 1, a signal detection 116 may include synthetic RNA motif, exogenous RNA motif, spike protein derived sequence, poly-A tail irregularities, and synthetic 5′ cap analog. A “synthetic RNA motif” as used in this disclosure, is a designed sequence or structural pattern added to laboratory made RNA to give it special behaviors that natural RNA might not have. A synthetic RNA motif may be human designed rather than naturally occurring and may be inserted intentionally into an RNA molecule. A synthetic RNA motif may be used to control stability, translation, folding or immune response.

With continued reference to FIG. 1, an “exogenous RNA motif” as used in this disclosure, is an RNA pattern or structure that comes from outside a cell or organism. An exogenous RNA motif may originate from viruses, bacteria, synthetic RNA, and/or environmental sources. For example, an exogenous RNA motif may include 5′-triphosphate RNA, double-stranded RNA (dsRNA), unmethylated CpG motifs, hairpin structures, and/or Poly-U or poly-I:C sequences.

With continued reference to FIG. 1, a “spike protein derived sequence” as used in this disclosure, is any amino acid sequence or nucleotide sequence that comes from the SARS-COV-2 spoke protein. A spike protein derived sequence may include a protein level spike derived sequence which may include peptides cut from the full spike protein. A spike protein derived sequence may include an RNA or DNA sequence that encodes part of the spike protein.

With continued reference to FIG. 1, a “poly-A tail irregularity” as used in this disclosure, is any abnormality of the length, structure, and/or placement of the poly-A tail on an mRNA molecule. The poly-A tail may include a long stretch of adenine nucleotides added to the 3′ end of eukaryotic mRNA to stabilize it, help export it from the nucleus, and regulate translation. An mRNA may have a tail of approximately 200-250 adenines. A poly-A tail irregularity may include an abnormal tail length such as if the tail is too short or too long. For example, a short poly-A tail may lead to rapid degradation because the tail may act as a protective buffer. In yet another non-limiting example, a long poly-A tail may trap mRNA in the nucleus instead of allowing export. A poly-A tail irregularity may include an incorrect polyadenylation site where mRNA may be cleaved at the wrong location before the tail is added. This may lead to abnormal transcripts or change protein output. A poly-A tail irregularity may include a faulty or incomplete tail addition such as when Poly-A polymerase may fail to add a proper tail. A poly-A tail irregularity may include an irregular tail composition where the tail may include other nucleotides or mixed sequences which are abnormal and disrupt binding by poly-A binding proteins (PABPs) which are essential for translation and stability. A poly-A tail irregularity may include a structural abnormality where the tail may fold or bind incorrectly if proteins fail to attach properly.

With continued reference to FIG. 1, a signal detection 116 may quantify a signal based molecular aberration contained in the RNA-sequencing data 112. A signal detection 116 may be utilized to assess post-COVD, post-vaccine, and/or chronic instability states. A signal detection 116 may be unique to a particular cancer, an autoimmune condition, a neurodegenerative disease, and/or an inflammatory disease. In an embodiment, a signal detection 116 may identify aberrations in immune regulation including for example cytokine signaling, antigen presentation, and/or TLR balance. A signal detection 116 may identify core hallmark expression genes and/or proteins involved in homeostatic regulation. A signal detection 116 may identify inflammatory resolution proteins that are misfiring such as NF-kB, STAT3, and IL6 pathway components. A signal detection 116 may identify structural signaling collagens, laminins that show downregulation or mistranslation such as fibrous clots and tissue strands. A signal detection 116 may identify ribosomal instability which contributes to mistranslation and misfolding. A signal detection 116 may identify dysregulation of TP53, BRCA½, RB1, and other key regulators. A signal detection 116 may identify activation of proliferation and migration such as MYC, KRAS, and BCL2. A signal detection 116 may identify spike-linked synthetic elements and/or contaminants such as Poly A, mt 12, Globin fragments, and foreign plasmids. A signal detection 116 may often be evidenced before symptomatic disease presents. A signal detection 116 demonstrating immune deregulation and oncogene instability may initiate chronic disease cascades.

With continued reference to FIG. 1, the at least a processor is configured to screen the signal detection 116 for an expression index 120. An “expression index” as used in this disclosure is an indication of the likelihood of transcriptomic profile 124 driving tumorigenesis. A “transcriptomic profile” as used in this disclosure, is a full analysis of the full set of RNA transcripts contained in the RNA-sequencing data 112. A transcriptomic profile 124 may include messenger RNA (mRNA) which carries instructions to make proteins. A transcriptomic profile 124 may include non-coding RNAs such as microRNAs and long non-coding RNAs that regulate gene expression. A transcriptomic profile 124 may indicate gene expression patterns, cell identity and function, indicators of how cells respond to disease, stress or treatment, and differences between health and diseased tissues. A transcriptomic profile 124 may be produced using one or more methodologies, including but not limited to microarrays, expressed sequence tags (ESTs), and/or serial analysis of gene expression (SAGE). A transcriptomic profile 124 may identify bulk transcriptomics indicating average gene expression across many cells; single cell transcriptomics indicating gene expression in individual cells; spatial transcriptomics which map gene expression while preserving tissue structure; and meta-transcriptomics which study RNA from communities such as the microbiome.

With continued reference to FIG. 1, “tumorigenesis” as used in this disclosure, is any indication of a health cell transforming into a tumor cell. This may include any step by step progression from a healthy cell to a mutated cell, to a precancerous lesion, and eventually to the formation of a malignant tumor. A tumorigenesis may involve genetic mutations, epigenetic alterations, microenvironment changes, and/or clonal expansion. Tumorigenesis may include initiation when a healthy cell acquires a mutation caused by a DNA replication error, an environmental exposure, chronic inflammation, and/or a viral infection. The mutated cell may then gain a growth advantage and outcompete neighboring cells. Additional mutations or environmental pressures push the cell toward uncontrolled growth and eventually cells acquire malignant traits such as invasion, immune evasion, angiogenesis, and/or metastatic potential. Tumorigenesis may include an identification of the driver of tumorigenesis including but not limited to oncogenes, tumor suppressor genes, DNA methylation changes, histone modifications, chromatin remodeling, chronic inflammation, immune dysfunction, fibroblast activation, and/or extracellular matrix stiffening.

With continued reference to FIG. 1, a “tumorigenesis likelihood” is an indication of the likelihood or probability of that a cell, tissue, organism, and/or human subject will develop a tumor based on one or more datapoints such as the transcriptomic profile 124, the RNA-sequencing data 112, the signal detection 116, genetic factors, environmental exposures, cellular microenvironment, immune surveillance and/or the expression index 120. The tumorigenesis likelihood 128 may indicate a probability of how likely a cell may accumulate cancer-driving mutations; how likely those mutations are to escape repair; how likely the microenvironment will support tumor growth; and/or how likely the immune system will fail to eliminate abnormal cells. A tumorigenesis likelihood 128 indicates the probability that tumor formation will occur, based on genetic, environmental, and biological factors. A tumorigenesis likelihood 128 may be reported a raw score indicating the number of points earned. A tumorigenesis likelihood 128 may be reported as a percentage score such as when a raw score is converted into a percentage. A tumorigenesis likelihood 128 may be reported as a scaled score where a raw score is fit to a standardized scale so different test forms or administrations can be compared. A tumorigenesis likelihood 128 may be reported as a percentile rank where different percentages of human subjects who score below a given score are reflected. A tumorigenesis likelihood 128 may be reported as a letter grade whereby scores may be grouped into categories such as A, B, C, etc. A tumorigenesis likelihood 128 may be reported as a criterion referenced score where scores are interpreted against fixed standards. A tumorigenesis likelihood 128 may be reported as a norm-referenced score where scores are interpreted relative to other human subjects. A tumorigenesis likelihood 128 may be reported as a graphical output such as a bar graph, a line graph, a scatter plot, and/or a pie chart. A tumorigenesis likelihood 128 may be reported as a table. A tumorigenesis likelihood 128 may be reported as a narrative description to contextualize numbers.

With continued reference to FIG. 1, a tumorigenesis likelihood 128 may be generated using a network activation mapping 132. A “network activation mapping” as used in this disclosure, is a process to visualize which parts of the input data activate specific neurons or layers of a machine learning process. A network activation mapping 132 may include a neural network process whereby information used to calculate a tumorigenesis likelihood 128 is processed through layers of neurons. Each neuron may respond or activate when it detects certain patterns. A network activation mapping may identify where activations occur, how strongly they occur, and which features of the input triggered them. A network activation mapping 132 may include but is not limited to techniques such as CAM (Class Activation Mapping), Grad-CAM, integrated gradients, saliency maps, and feature visualization. A network activation mapping 132 may include feeding an input into the neural network; recording neuron activations at chosen layers; mapping those activations back onto the input space; and visualizing them as heatmaps or overlays. A network activation mapping 132 may be visualized as a heatmap, a color overlay, a feature map, and/or a layer activation grid. A network activation mapping 132 may appear with a token level importance score, a sequence heatmap, and/or a feature importance plot.

With continued reference to FIG. 1, the at least a professor may generate a transcriptomic profile 124 using the RNA-sequencing data 112 and the signal detection 116 and identify a tumorigenesis likelihood 128 using an oncogene library 136. An “oncogene library” as used in this disclosure, is a collection of oncogenes and/or pro-oncogenes. An oncogene library 136 may be organized as a database 140. An oncogene library 136 may contain known human oncogenes, mutant variants of proto-oncogenes, viral oncogenes, and engineered oncogenes. The oncogene library 136 may be sourced from cancer genomics databases such as The Cancer Genome Atlas (TCGA), TARGET, CPTAC, CGCI, HCMI and other NCI-funded programs. The oncogene library 136 may contain data and research from COSMIC cancer gene census, and OncoKB cancer gene list. The oncogene library 136 may contain data from multi-omics integrative platforms such as OncoDB. The oncogene library 136 may contain data from cancer research consortia and data commons such as GDC, PDC, and ICDC.

With continued reference to FIG. 1, a “database 140” as used in this disclosure, is an organized collection of data and information that can be stored, searched, updated, and retrieved efficiently. A database 140 may include data entries and a structure that contains rules indicating how data is organized. A database 140 may include a query system that allows the at least a processor 104 to ask questions about the data entries. A database 140 may contain controls which include permissions, security, and backups. A database 140 may include but is not limited to a relational database 140, a NoSQL database 140, a graph database 140, a time series database 140, and a document database 140.

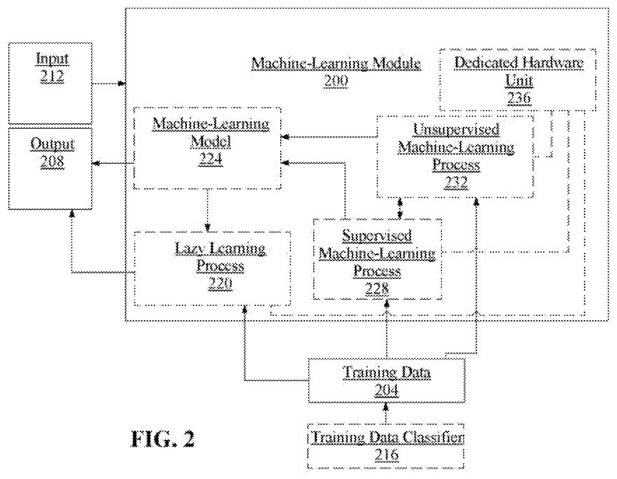

With continued reference to FIG. 1, network activation mapping 132 may include generating a large language model. Large language model may include any large language model as described below in more detail in reference to FIG. 2.

With continued reference to FIG. 1, the at least a processor 104 is configured to calculate a risk score 144 using the RNA-sequencing data 112, the signal detection 116, the transcriptomic profile 124, the tumorigenesis profile, the tumorigenesis likelihood 128 and the expression index 120. A “risk score” as used in this disclosure, is an indicator of a quantitative score reflecting biological risk related to the human subject. A risk score 144 may contain a numerical value representing biological risk related to the human subject. The risk score 144 may contain a calculated number reflecting the level of risk for the human subject. The risk score 144 may reflect a likelihood representing how probable an event is. The risk score 144 may reflect an impact representing how damaging the event would be if it occurred. The risk score 144 may represent a combination of both the likelihood and the impact based on weighing various factors utilized to calculate the risk score 144, including the RNA-sequencing data 112, the signal detection 116, and the expression index 120. The risk score 144 may aid in prioritizing which risks need attention first, support decision making and resource allocation, track changes in risk over time, and standardize risk assessment across human subjects.

With continued reference to FIG. 1, “biological risk” as used in this disclosure, is the chance that exposure to an agent will cause harm. Biological risk may reflect the likelihood that bacteria, viruses, fungi, parasites, biological toxins, vaccines, medications, treatments and the like may lead to infection, illness, and/or other negative health effects. An agent may include disease producing agents such as pathogens which can be transmitted through various routes and may cause acute or chronic health conditions. An agent may include pathogens transmitted through direct or indirect exposure, increasing the risk of infection. An agent may include biological substances that threaten the health of a living organism. Biological risk may be calculated as a probability multiplied by a severity of harm from a biological agent. A biological risk may depend on the type of agent, how much exposure occurs, the route of exposure, the vulnerability of the exposed person, and environmental conditions. A biological risk may include for example, bloodborne pathogens, airborne pathogens, microbiological waste, human or animal fluids, molds, yeasts, fungi, biological toxins, contaminated sharps, organic waste containing pathogens, abrin, aerolysin, Botulinum Toxin A, Botulinum Toxin B, Botulinum Toxin C1, Botulinum Toxin C2, Botulinum Toxin D, Botulinum Toxin E, Botulinum Toxin F, B-Bungarotoxin, Caeruleotoxin, Cereolysin, Ciguatoxin 2, Ciguatoxin 3, Clostridium difficile enterotoxin A, Clostridium perfringens alpha toxin, lecithinase, Clostridium perfringens theta toxin, perfringolysin O, Clostridium perfringens enterotoxin, Clostridium perfringens beta-toxin, Clostridium perfringens dela-toxin, Clostridium perfringens epsilon-toxin, Conotoxins, Crotoxin, Diacetoxyscirpenol (DAS), Diphtheria toxin, Listeriolysin, Maitotoxin, Modeccin, Nematocyst toxins, Notexin, Palytoxin, Pertussis toxin, Pneumolysin, Pseudomonas aeruginosa exotoxin A, Ricin, Saxitoxin, Scaritoxin, Shiga toxin, Shigella dysenteriae neurotoxin, Streptolysin O, Streptolysin S, Staphylococcus aureus alpha toxin, Staphylococcus aureus enterotoxin B, Staphylococcus aureus entertoxin F, T-2 toxin, Taipoxin, Tetanus toxin, Tetrodotoxin, Viscumin, Volkensin, Yersinia pestis murine toxin, thimerosal, aluminum, adjuvants, antigen, preservatives, formaldehyde, gelatin, stabilizers, antibiotics, polysorbate 80, genetically modified organisms (GMOs), DNA fragments, prions, Vitamin A, spike protein, shedding, stabilizers, trace components, excipients, polyethylene glycol, preservatives, heavy metals, vaccines including but not limited to Hepatitis B vaccine, diphtheria, pertussis, and tetanus (DPT) vaccine, flu shot, pneumococcal conjugate vaccines, human papillomavirus (HPV) vaccines, COVID-19 vaccines and boosters, mRNA vaccines, polio (IPV), hepatitis A, varicella (chickenpox), yellow fever, shingles (Shingrix), viral vector vaccines, yellow fever, typhoid, Japanese encephalitis, and the like.

With continued reference to FIG. 1, the risk score 144 may be calculated using a machine learning model 152, including any machine learning model as described herein. Calculating the risk score 144 may include training a machine learning model using training data 148, wherein the training data 148 comprises a plurality of RNA sequencing data relating to human subjects, signal detection 116, expression index 120es, and risk score 144. “Training data” as used herein, is data containing correlations that a machine learning model 152 and/or process may use to model relationships between two or more categories of data elements. For instance and without limitation, training data 148 may include a plurality of data entries, also known as “training examples,” each entry representing a set of data elements that were recorded, received, and/or generated together; data elements may be correlated by shared existence in a given data entry, by proximity in a given data entry, or the like. Training data 148 may include any training data suitable for use as training data 204 as described below in more detail in reference to FIG. 2. Training data 148 may be sourced from open and public datasets, government data portals, commercial data providers, internal or proprietary data, crowdsourced data, synthetic data, web-scraped or internet scale data, domain specific repositories, and the like. For instance and without limitation, training data 148 may be obtained from genomics and sequencing database 140 such as 1000 Genomes Project, ENCODE, GEO (Gene Expression Omnibus), ArrayExpress, UK Biobank and the like. In yet another non-limiting example, training data 148 may be obtained from cancer and disease focused repositories such as The Cancer Genome Atlas (TCGA), Cancer Cell Line Encyclopedia (CCLE), METABRIC, Human Cancer Models Initiative (HCMI) and the like. In yet another non-limiting example, training data 148 may be obtained from imaging and microscopy data such as Allen Brain Observatory and Allen Cell Imaging Collections, Cell Painting Image Collection, The Cancer Imaging Archive (TCIA), Electron microscopy datasets (COSEM) and the like. In yet another non-limiting example, training data 148 may be obtained from single cell and multi-omics repositories including for example, Single Cell Expression Atlas, scPerturb, and Tabula Sapiens and Tabula Muris Senis. In yet another non-limiting example, training data 148 may be obtained from clinical and health data such as the National Institute of Health (NIH) All of Us Research Program, electronic health records (EHRs), clinical trial sequencing projects and the like. In yet another non-limiting example, training data 148 may be obtained from experimental and lab generated data including sequencing instruments, high resolution microscopes, flow cytometry, proteomics and metabolomics assays, and environmental and ecological surveys. Training data 148 may be obtained from synthetic and simulation based data such as synthetic antibody-antigen binding datasets, simulated genomes, protein structures, and cell images, and in silico perturbation datasets.

With continued reference to FIG. 1, the at least a processor 104 may assign a weight to each of the RNA-sequencing data 112 related to the human subject, the signal detection 116, and the expression index 120. A “weight” as used in this disclosure, is a numerical value that denotes how strongly an input influences a model's prediction. A weight may be a learned parameter that scales an input feature. In an embodiment, a high weight may indicate a strong influence on the output whereby a lower weight may indicate a weaker influence. During training, the machine learning model 152 may adjust its weights to reduce error. Weights may be determined and assigned based upon expert input. Expert input may include information, guidance, and knowledge that may be obtained from scientists, researchers, clinicians, medical specialists, engineers, technical experts, domain specific professionals, and experienced practitioners with tacit knowledge. For example, expert input may include labeling cell types in RNA-sequencing data 112, annotating pathology images, defining biological pathways or gene sets, interpreting variant pathogenicity, designing training labels for disease classification, and setting quality control thresholds for sequencing data.

With continued reference to FIG. 1, a “machine learning model” as used in this disclosure, is a process that automatedly uses training data 148 to generate an algorithm instantiated in hardware or software logic, data structures, and/or functions that will be performed by the processor 104 to produce outputs given data provided as inputs; this is in contrast to a non-machine learning software program where the commands to be executed are determined in advance by a user and written in a programming language. Machine learning model 152 may be implemented as any machine learning process as described below in more detail in reference to FIG. 2.

With continued reference to FIG. 1, the weighted RNA-sequencing data 112 related to the human subject, the signal detection 116, and the expression input may be input into the trained machine learning model 152. The machine learning model 152 may output the risk score 144 from the trained machine learning model 152.

With continued reference to FIG. 1, the risk score 144 may be stratified as Normal (0-10), Mild (10-25), Moderate (25-40), and High (>40). The risk score 144 may be displayed as a graphical representation for continued monitoring of the human subject. This may include visual displays including bar graphs, pie charts, and timeline progression charts for ongoing monitoring of individual patients.

With continued reference to FIG. 1, the at least a processor 104 may include a graphical user interface (GUI 156) that may be utilized to display the risk score 144 and the graphical representation of the risk score 144. A “GUI” as used in this disclosure, is a visual layer that allows a user to interact with the GUI 156 and the at least a processor 104 using buttons, icons, menus, windows, and other graphical elements. A GUI 156 may include buttons, icons, menus and dropdowns, text boxes, forms, sliders and toggles, windows and panels, graphs and visual displays. A GUI 156 may be utilized to visualize gene expression, browse sequencing reads, explore microscopy images, and interact with complex models or datasets.

With continued reference to FIG. 1, the GUI 156 may display an intervention based on the risk score 144. An “intervention” as used in this disclosure is any recommended action to change, improve, or influence a risk score 144. An intervention may include a prescription medication, a supplement, a nutraceutical, a lifestyle change, a diagnostic test used to GUI 156de treatments, individualized peptide sequences tailored to a patient's genetic risk factors, expressed molecular signatures, and immune system profile, and the like. An intervention may target molecular dysfunction as evidenced by a risk score 144, including but not limited to cancer, autoimmune disease, neurodegenerative disease, environmental or viral-triggered illness, Lyme associated illness and the like.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example inputs may include epidemiological and/or genomic data, such as transcriptomic data and outputs may include observed patterns and predictions of the data.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to various sub-cohorts such as mutation versus normal.

Still referring to FIG. 2, a computing device may be configured to generate a classifier using a Naive Bayes classification algorithm. Naive Bayes classification algorithm generates classifiers by assigning class labels to problem instances, represented as vectors of element values. Class labels are drawn from a finite set. Naive Bayes classification algorithm may include generating a family of algorithms that assume that the value of a particular element is independent of the value of any other element, given a class variable. Naive Bayes classification algorithm may be based on Bayes Theorem expressed as P(A/B)=P(B/A) P(A)±I³(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naive Bayes algorithm may be generated by first transforming training data into a frequency table. Computing device may then calculate a likelihood table by calculating probabilities of different data entries and classification labels. A computing device may utilize a naive Bayes equation to calculate a posterior probability for each class. A class containing the highest posterior probability is the outcome of prediction. Naive Bayes classification algorithm may include a gaussian model that follows a normal distribution. Naive Bayes classification algorithm may include a multinomial model that is used for discrete counts. Naive Bayes classification algorithm may include a Bernoulli model that may be utilized when vectors are binary.

With continued reference to FIG. 2, a computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements. With continued reference to FIG. 2, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute 1 as derived using a Pythagorean norm: 1=aᵢ2, where a, is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. A computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 2, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 200 pixels, however a desired number of pixels may be 228. Processor may interpolate the low pixel count image to convert the 200 pixels into 228 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units. In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 2, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 2, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value xₘᵢₙ in a set or subset of values subtracted therefrom, with the result divided by the range of the values, give maximum value in the set or subset xₘₐₓ:

$${x}_{new} \frac{x - x min}{x max - x min}$$

Feature scaling may include mean normalization, which involves use of a mean value of a set and/or subset of values, xₘₑₐₙ with maximum and minimum values

$${x}_{new} \frac{x - x mean}{x max - x min}$$

Feature scaling may include standardization, where a difference between X and Xₘₑₐₙ is divided by a standard deviation a of a set or subset of values:

$$Xnew = \frac{x - {x}_{mean}}{a}$$

Scaling may be performed using a median value of a set or subset x_{median} and/or interquartile range (IQR), which represents the difference between the 25^{th} percentile value and the 50^{th} percentile value (or closest values thereto by a rounding protocol), such as:

$${x}_{new} \frac{x - x median}{IQR} .$$

persons skilled in the art, upon reviewing the entirety of this disclosure, will be aware of various alternative or additional approaches that may be used for feature scaling.

Further referring to FIG. 2, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described above as inputs, outputs described above as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 2 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Still referring to FIG. 2, machine-learning module 200 may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, nonlimiting examples, medical report documents, electronic health records, entity documents, business documents, inventory documentation, emails, user communications, advertising documents, newspaper articles, and the like. In some embodiments, training sets of an LLM may include information from one or more public or private databases. As a non-limiting example, training sets may include databases associated with an entity. In some embodiments, training sets may include portions of documents associated with the electronic records correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 2, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 2, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 2, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 2, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 2, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 2, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bandanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 2, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. A query vector may include an entity's learned representation for comparison to determine attention score. A key vector may include an entity's learned representation for determining the entity's relevance and attention weight. A value vector may include data used to generate output representations. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 2, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 2, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 2, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 2, in some embodiments, input to decoder may go through an embedding layer and positional encoding layer in order to obtain positional embeddings. Decoder may include a first multi-headed attention layer, wherein the first multiheaded attention layer may receive positional embeddings.

With continued reference to FIG. 2, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “Os” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 2, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 2, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

In various embodiments, lipid nanoparticles may further comprise optional targeting ligands (for cell-specific delivery), including but are not limited to: Monoclonal antibodies, antibody fragments, receptor-binding peptides, aptamers, GalNAc, mannose, RGD peptides, small molecule ligands.

Still referring to FIG. 2, decoder may take this output and add it to the decoder inputs. Decoder may continue decoding until a token is predicted. Decoder may stop decoding once it predicts an end token.

Continuing to refer to FIG. 2, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 2, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with transcriptomic data.

With continued reference to FIG. 2, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

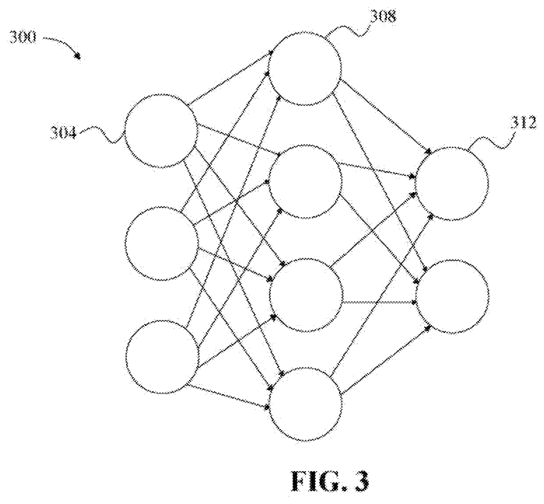

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

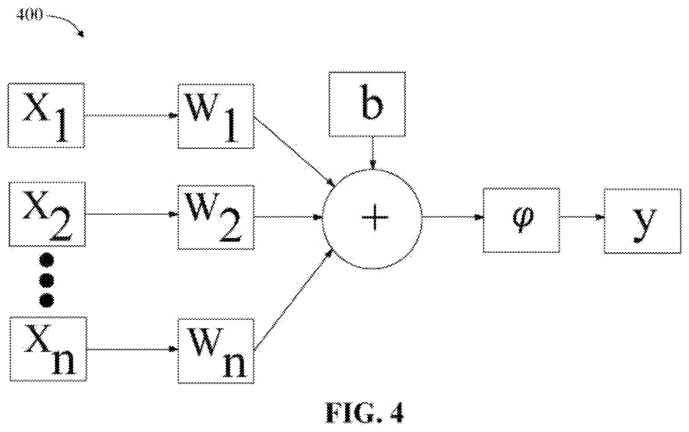

Referring now to FIG. 4, an exemplary embodiment of a node 400 of a neural network is illustrated. A node may include, without limitation a plurality of inputs x, that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = = \frac{1}{1 - {e}^{- x}} x$$

given input x, a tanh (hyperbolic tangent) function, of the form

$$\frac{{e}^{x} - {e}^{- x}}{{e}^{x} + {e}^{- x}}$$

a tanh derivative function such as ƒ(x)=tanh²(x), a rectified linear unit function such as ƒ(x)=max (0, x), a “leaky” and/or “parametric” rectified linear unit function such as ƒ(x)=max (ax, x) for some a, an_x for x>0_exponential linear units function such as

$$f ( x ) \{ \begin{matrix}x for x \geq 0 \\ α ( {e}^{x} - 1 ) for x < 0\end{matrix}$$

for some value of a_(this function may be replaced and/or weighted by its own derivative in some embodiments), a softmax function such as

$$f ( {x}_{i} ) = \frac{{e}^{x}}{\sum ixi}$$

where the inputs to an instant layer are xᵢ, a swish _{Li}xᵢ function such as ƒ(x)=x*sigmoid(x), a Gaussian error linear unit function such as f(x)=ƒ(x)=a(1+tanh (\sqrt{2/π}(x+bx^{r}))) for some values of a, b, and r, and/or a scaled exponential linear unit function such as

$$f ( x ) = f ( x ) \{ \begin{matrix}α ( {e}^{x} - 1 ) for x < 0 \\ x for x \geq 0\end{matrix} .$$

Fundamentally, there is no limit to the x for x>0_nature of functions of inputs x, that may be used as activation functions. As a non-limiting and illustrative example, node may perform a weighted sum of inputs using weights wi that are multiplied by respective inputs z. Additionally or alternatively, a bias b may be added to the weighted sum of the inputs such that an offset is added to each unit in the neural network layer that is independent of the input to the layer. The weighted sum may then be input into a function co, which may generate one or more outputs y. Weight wi applied to an input x, may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wi may be determined by training a neural network using training data, which may be performed using any suitable process as described above.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission. Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

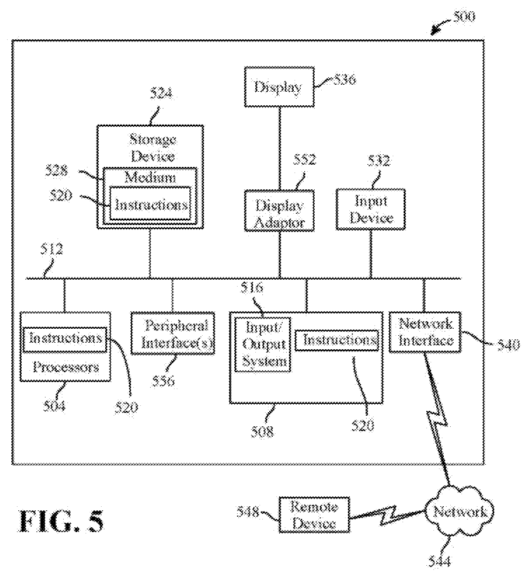

FIG. 5 shows a diagrammatic representation of one embodiment of a computing device in the exemplary form of a computer system 500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 500 includes a processor 504 and a memory 508 that communicate with each other, and with other components, via a bus 512. Bus 512 may include any of several types of bus structures including, but not limited to, a memory bus, a memory controller, a peripheral bus, a local bus, and any combinations thereof, using any of a variety of bus architectures.

Processor 504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 504 may be organized according to Von Neumann and/or Harvard architecture as a non-limiting example. Processor 504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 508 may include various components (e.g., machine-readable media) including, but not limited to, a random-access memory component, a read only component, and any combinations thereof. In one example, a basic input/output system 516 (BIOS), including basic routines that help to transfer information between elements within computer system 500, such as during start-up, may be stored in memory 508. Memory 508 may also include (e.g., stored on one or more machine-readable media) instructions (e.g., software) 520 embodying any one or more of the aspects and/or methodologies of the present disclosure. In another example, memory 508 may further include any number of program modules including, but not limited to, an operating system, one or more application programs, other program modules, program data, and any combinations thereof.

Computer system 500 may also include a storage device 524. Examples of a storage device (e.g., storage device 524) include, but are not limited to, a hard disk drive, a magnetic disk drive, an optical disc drive in combination with an optical medium, a solid-state memory device, and any combinations thereof. Storage device 524 may be connected to bus 512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 524 (or one or more components thereof) may be removably interfaced with computer system 500 (e.g., via an external port connector (not shown)). Particularly, storage device 524 and an associated machine-readable medium 528 may provide nonvolatile and/or volatile storage of machine-readable instructions, data structures, program modules, and/or other data for computer system 500. In one example, software 520 may reside, completely or partially, within machine-readable medium 528. In another example, software 520 may reside, completely or partially, within processor 504.

Computer system 500 may also include an input device 532. In one example, a user of computer system 500 may enter commands and/or other information into computer system 500 via input device 532. Examples of an input device 532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 532 may be interfaced to bus 512 via any of a variety of interfaces (not shown) including, but not limited to, a serial interface, a parallel interface, a game port, a USB interface, a FIREWIRE interface, a direct interface to bus 512, and any combinations thereof. Input device 532 may include a touch screen interface that may be a part of or separate from display 536, discussed further below. Input device 532 may be utilized as a user selection device for selecting one or more graphical representations in a graphical interface as described above.

A user may also input commands and/or other information to computer system 500 via storage device 524 (e.g., a removable disk drive, a flash drive, etc.) and/or network interface device 540. A network interface device, such as network interface device 540, may be utilized for connecting computer system 500 to one or more of a variety of networks, such as network 544, and one or more remote devices 548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 544, may employ a wired and/or a wireless mode of communication. In general, any network topology may be used. Information (e.g., data, software 520, etc.) may be communicated to and/or from computer system 500 via network interface device 540.

Computer system 500 may further include a video display adapter 552 for communicating a displayable image to a display device, such as display device 536. Examples of a display device include, but are not limited to, a liquid crystal display (LCD), a cathode ray tube (CRT), a plasma display, a light emitting diode (LED) display, and any combinations thereof. Display adapter 552 and display device 536 may be utilized in combination with processor 504 to provide graphical representations of aspects of the present disclosure. In addition to a display device, computer system 500 may include one or more other peripheral output devices including, but not limited to, an audio speaker, a printer, and any combinations thereof. Such peripheral output devices may be connected to bus 512 via a peripheral interface 556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and compositions according to the present disclosure. Accordingly, this description is meant to be taken only by way of example, and not to otherwise limit the scope of this invention. Exemplary embodiments have been disclosed above and illustrated in the accompanying drawings. It will be understood by those skilled in the art that various changes, omissions and additions may be made to that which is specifically disclosed herein without departing from the spirit and scope of the present invention.

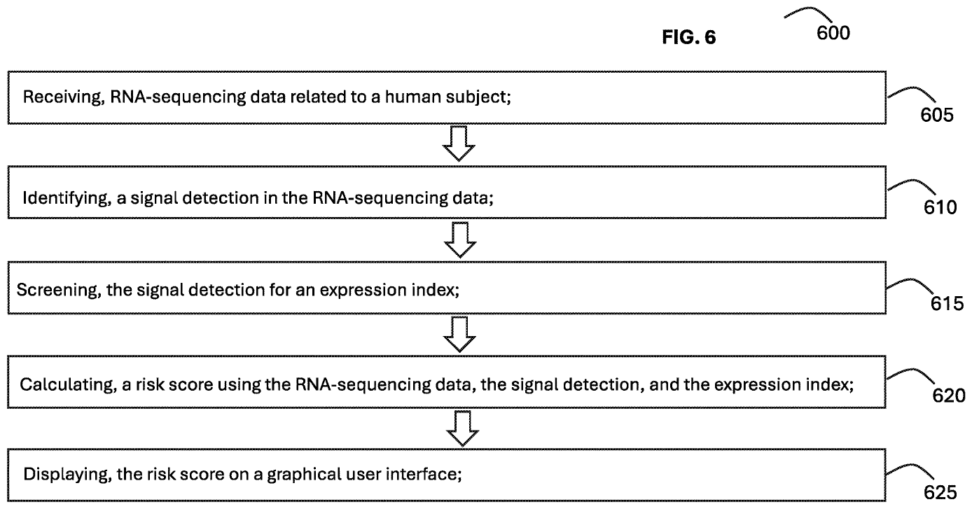

Referring now to FIG. 6, an exemplary embodiment of a method 600 for analyzing RNA-sequencing (RNA-seq) data to detect expression aberrations and synthetic RNA interference is illustrated. At step 605, at least a processor 104 receives RNA-sequencing data 112 related to a human subject. RNA-sequencing data 112 may include any RNA-sequencing data 112 as described above in more detail in reference to FIG. 1. RNA-sequencing data 112 may be selected from a group consisting of immune regulation, mitochondrial function, cell cycle, inflammation, and DNA repair.

With continued reference to FIG. 6, at step 610, the at least a processor 104 identifies a signal detection 116 in the RNA-sequencing data 112. A signal detection 116 includes any signal detection 116 as described above in more detail in reference to FIG. 1. The signal detection 116 may consist of synthetic RNA motif, exogenous RNA motif, spike protein derived sequence, poly-A tail irregularities, and synthetic 5′ cap analog.

With continued reference to FIG. 6, at step 615, the at least a processor 104 screens the signal detection 116 for an expression index. The expression index 120 includes any expression index 120 as described above in more detail in reference to FIG. 1. The expression index 120 may include generating a transcriptomic profile 124 using the RNA-sequencing data 112 and the signal detection 116 and identifying a tumorigenesis likelihood 128 using an oncogene library 136. This may be performed utilizing any methodology as described above in more detail in reference to FIG. 1 The tumorigenesis likelihood 128 may be identified using a network activation network. This may include generating a large language model (LLM) as described above in more detail in reference to FIGS. 1-5.

With continued reference to FIG. 6, at step 620, the at least a processor calculates a risk score 144 using the RNA-sequencing data 112, the signal detection 116, and the expression index. The risk score 144 includes any risk score 144 as described above in more detail in reference to FIGS. 1-5. The risk score 144 may be calculated using a machine learning process. This may include training a machine learning model 152 using training data 148, wherein the training data 148 set comprises a plurality of RNA-sequencing data 112 relating to human subjects, signal detection 116, expression indexes, and risk score 144. The at least a processor 104 may assign a weight to each of the RNA-sequencing data 112 related to the human subject, the signal detection 116, and the expression index. The weighted RNA-sequencing data 112 related to the human subject, the signal detection 116, and the expression index 120 may be input into the trained machine learning model 152. The risk score 144 may be output from the trained machine learning model 152.

With continued reference to FIG. 6, at step 625, the at least a processor 104 displays the risk score 144 on a graphical user interface. The risk score 144 may be selected from a group consisting of normal, mild, moderate, and high. The risk score 144 may be displayed on the GUI 156 for continued monitoring of the human subject. In an embodiment, an intervention based on the risk score 144 may be displayed on the GUI 156. The intervention may include any of the interventions as described above in more detail in reference to FIGS. 1-5.

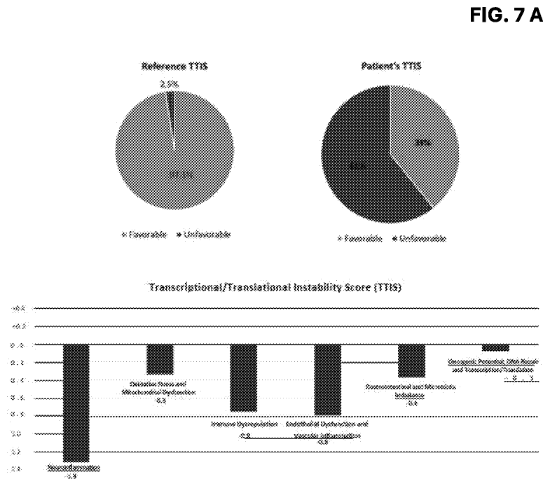

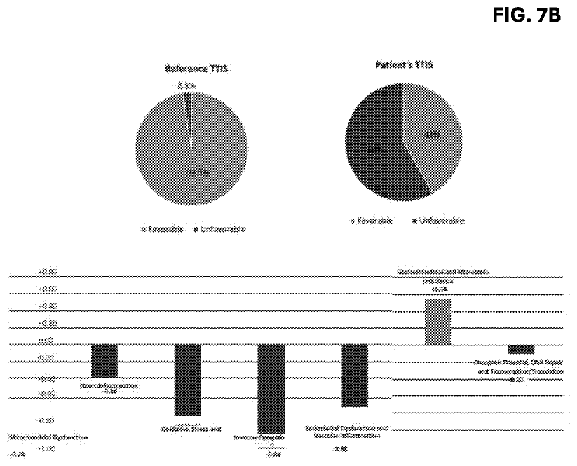

Referring now to FIG. 7A, an exemplary embodiment 700 of a transcriptomic profile 124 is illustrated. In an embodiment, a transcriptomic profile 124 may reflect a favorable or unfavorable measurement. A favorable measurement may indicate a transcriptomic profile 124 that contains advantageous and/or beneficial gene expression. An unfavorable measurement may indicate a transcriptomic profile 124 that contains disadvantageous and/or negative gene expression. A transcriptomic profile 124 relating to a human subject may be compared and evaluated in reference to a control or reference transcriptomic profile 124. In an embodiment, a transcriptomic profile 124 may contain a breakdown of gene expression relating to certain body system and gene groupings such as neuroinflammation, oxidative stress and mitochondrial dysfunction, immune dysregulation, endothelia dysfunction and vascular inflammation, gastrointestinal and microbiota imbalance, and oncogenic potential, DNA repair and transcription and translation. Referring now to FIG. 7B, an exemplary embodiment of a transcriptomic profile 124 is illustrated. The transcriptomic profile 124 may include a breakdown calculated as a percentage of favorable and unfavorable gene expression. This may be compared to a reference subject. The transcriptomic profile 124 may include a breakdown by gene expression for certain groupings such as neuroinflammation, oxidative stress and mitochondrial dysfunction, immune dysregulation, endothelial dysfunction and vascular inflammation, gastrointestinal and microbiota imbalance, and oncogenic potential, DNA repair and transcription and translation. In an embodiment, this may be displayed on GUI 156 as a bar graph with a positive number indicating favorable gene expression and a negative number indicating an unfavorable gene expression.

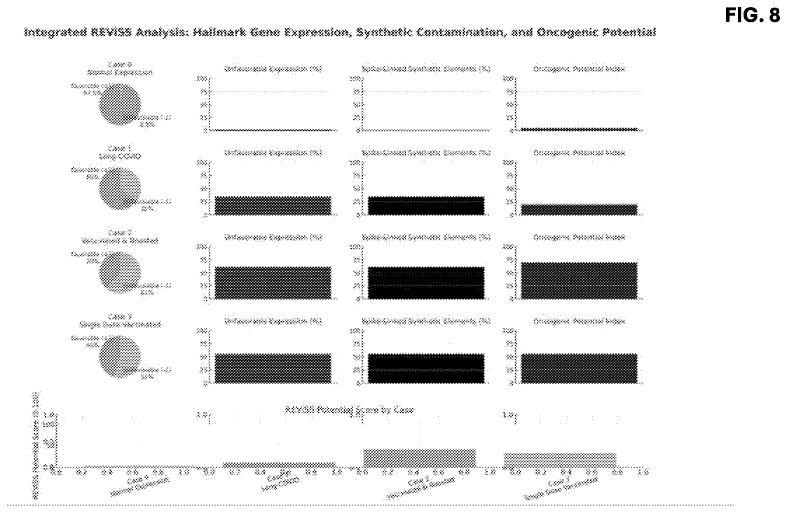

Referring now to FIG. 8, an exemplary embodiment 800 of risk score 144 is illustrated. Risk score 144 may be displayed showing favorable and unfavorable expression, spike-linked synthetic elements, and oncogenic potential index. Depending on a human subject's individual circumstances, gene expression, synthetic contamination, and oncogenic potential may look differently. In an embodiment, a score for each use case may be calculated and displayed on GUI 156. For example, Case 0 demonstrates normal expression; Case 1 demonstrates long-COVID, Case 2 demonstrates vaccinated & boosted, and Case 3 demonstrates single dose vaccinated. In an embodiment, risk score 144 may be displayed on GUI 156 as a graphical representation to track changes over time.

What is claimed is:

1. A system for analyzing RNA-sequencing (RNA-seq) data to detect expression aberrations and synthetic RNA interference, the system comprising:
at least a processor; and
a memory communicatively connected to the at least a processor, the memory containing instructions configuring the at least a processor to:
receive RNA-sequencing data related to a human subject;
identify a signal detection in the RNA-sequencing data;
screen the signal detection for an expression index;
calculate a risk score using the RNA-sequencing data, the signal detection, and the expression index; and
display, the risk score on a graphical user interface.

2. The system of claim 1, wherein the RNA-sequencing data is selected from a group consisting of: immune regulation, mitochondrial function, cell cycle, inflammation, and DNA repair.

3. The system of claim 1, wherein the signal detection is selected from a group consisting of: synthetic RNA motif, exogenous RNA motif, spike protein derived sequence, poly-A tail irregularities, and synthetic 5′ cap analog.

4. The system of claim 1, wherein the expression index further comprises:
generating, a transcriptomic profile using the RNA-sequencing data and the signal detection;
and identifying a tumorigenesis likelihood using an oncogene library.

5. The system of claim 4 further comprising identifying the tumorigenesis likelihood using a network activation mapping.

6. The system of claim 5, wherein the network activation mapping further comprises generating a large language model (LLM).

7. The system of claim 1, wherein calculating the risk score further comprises:
training a machine learning model using training data, wherein the training data comprises a plurality of RNA-sequencing data relating to human subjects, signal detections; expression indexes; and risk scores;
assigning a weight to each of the RNA-sequencing data related to the human subject, the signal detection, and the expression index;
inputting, the weighted RNA-sequencing data related to the human subject, the signal detection, and the expression index into the trained machine learning model; and
outputting, the risk score from the trained machine learning model.

8. The system of claim 1, wherein the risk score is selected from the group consisting of:
normal, mild, moderate, and high.

9. The system of claim 1, wherein the risk score is displayed as a graphical representation for continued monitoring of the human subject.

10. The system of claim 1 further comprising displaying, an intervention based on the risk score.

11. A method of analyzing RNA-sequencing (RNA-seq) data to detect expression aberrations and synthetic RNA interference, the method comprising:
receiving, by at least a processor, RNA-sequencing data related to a human subject;
identifying, by the at least a processor, a signal detection in the RNA-sequencing data;
screening, by the at least a processor, the signal detection for an expression index;
calculating, by the at least a processor, a risk score using the RNA-sequencing data, the signal detection, and the expression index; and
displaying, by the at least a processor, the risk score on a graphical user interface.

12. The method of claim 11, wherein the RNA-sequencing data is selected from a group consisting of: immune regulation, mitochondrial function, cell cycle, inflammation, and DNA repair.

13. The method of claim 11, wherein the signal detection is selected from a group consisting of: synthetic RNA motif, exogenous RNA motif, spike protein derived sequence, poly-A tail irregularities, and synthetic 5′ cap analog.

14. The method of claim 11, wherein the expression index further comprises:
generating, a transcriptomic profile using the RNA-sequencing data and the signal detection;
and identifying a tumorigenesis likelihood using an oncogene library.

15. The method of claim 14 further comprising identifying the tumorigenesis likelihood using a network activation mapping.

16. The method of claim 15, wherein the network activation mapping further comprises generating a large language model (LLM).

17. The method of claim 11, wherein calculating the risk score further comprises:
training a machine learning model using training data, wherein the training data set comprises a plurality of RNA-sequencing data relating to human subjects, signal detections; expression indexes, and risk scores;
assigning a weight to each of the RNA-sequencing data related to the human subject, the signal detection, and the expression index;
inputting, the weighted RNA-sequencing data related to the human subject, the signal detection, and the expression index into the trained machine learning model; and
outputting, the risk score from the trained machine learning model.

18. The method of claim 11, wherein the risk score is selected from the group consisting of: normal, mild, moderate, and high.

19. The method of claim 11, wherein the risk score is displayed as a graphical representation for continued monitoring of the human subject.

20. The method of claim 11 further comprising displaying, an intervention based on the risk score.
